(19) United States
(12) Patent Application Publication
(10) Pub. No.: US 2015/0306271 A1
(43) Pub. Date: Oct. 29, 2015

(54) ADSORPTIVE PHOTO-CATALYTIC OXIDATION AIR PURIFICATION DEVICE

(71) Applicant: Triatomic Environmental, Inc., Jupiter, FL (US)

(72) Inventor: Christopher C. Willette, Jupiter, FL (US)

(21) Appl. No.: 14/796,653

(22) Filed: Jul. 10, 2015

Related U.S. Application Data
(63) Continuation-in-part of application No. 13/870,752, filed on Apr. 25, 2013, now abandoned.
Application No. 13/870,752 is a continuation of application No. 12/793,328, filed on Jun. 3, 2010, now abandoned.
(60) Provisional application No. 61/183,614, filed on Jun. 3, 2009.

(51) Int. Cl. A61L 9/20

(52) U.S. Cl. CPC A61L 9/205

(57) ABSTRACT

An air purification system formed from an adsorptive photocatalytic oxidation device and a method of regenerating the oxidation device is disclosed. The air purification system may be configured to be installed within an air duct of a central air handling system. The air purification system may also include an ultraviolet light emitted by the ultraviolet light source to break down captured volatile organic compounds into elemental carbon dioxide and water vapor, and to irradiate air moving past the ultraviolet light and surfaces to reduce contaminants. The ultraviolet light source may be positioned to expose the adsorptive photocatalytic oxidation device to ultraviolet light emitted by the ultraviolet light source to break down captured volatile organic compounds into elemental carbon dioxide and water vapor, and to irradiate air moving past the ultraviolet light to reduce contaminants.

### CROSS REFERENCE TO RELATED APPLICATION

In accordance with 37 CFR 1.76, a claim of priority is included in an Application Data Sheet filed concurrently herewith. Accordingly, the present invention claims priority as a continuation in part of U.S. patent application Ser. No. 13/870,752, entitled, “Absorptive Photo-Catalytic Oxidation Air Purification Device”, filed Apr. 25, 2013, which is a continuation of U.S. patent application Ser. No. 12/793,328, entitled, “Absorptive Photo-Catalytic Oxidation Air Purification Device”, filed Jun. 3, 2010, which claims the benefit of U.S. Provisional Patent Application Ser. No. 61/183,614, filed Jun. 3, 2009. The contents of the above referenced applications are incorporated herein by reference in their entirety.

### FIELD OF THE INVENTION

The present invention is directed generally to air purification systems and devices, and more particularly, to air purification systems/devices for removal of volatile organic compounds or microorganisms.

### BACKGROUND

Recent studies have shown that the level of invisible airborne organic chemical and odor contaminates in our indoor air is generally two to five times higher than the levels found outdoors. These potentially harmful contaminates, known as volatile organic compounds (VOCs) are a large group of carbon-based chemicals that easily evaporate at room temperature. While most people can smell high levels of some volatile organic compounds, other volatile organic compounds have no odor. Odor does not indicate the level of risk from inhalation of this group of chemicals. There are thousands of different volatile organic compounds produced and used in our daily lives. Some common examples include: acetone, benzene, ethylene glycol, formaldehyde, methylene chloride, perchloroethylene, toluene and xylene. Volatile organic compounds are often released from products such as building materials, carpets, adhesives, upholstery fabrics, vinyl floors, composite wood products, paints, varnishes, sealing caulks, glues, carpet cleaning solvent, home care products, air fresheners, air cleaners that produce ozone, cleaning and disinfecting chemicals, cosmetics, smoking, fireplaces, fuel oil, gasoline, moth balls and vehicle exhaust from running a car in an attached garage. Daily activities that release volatile organic compounds include: cooking, dry cleaning clothes, carpet cleaning, household cleaning, hobbies, crafts, newspapers, magazines, non-electric space heaters, photocopiers, smoking, stored paints and chemicals, and wood burning stoves.

The health risks from inhaling any chemical depend on how much is in the air, and how long and how often a person inhales the chemical. Scientists look at short-term (acute) exposures as an exposure between a period of hours to a period of days, or long-term (chronic) exposures as years to even a lifetime. Breathing low levels of volatile organic compounds for long periods of time may increase the risk of health problems for some people. Several studies suggest that exposure to volatile organic compounds may make symptoms worse in people who have asthma or are particularly sensitive to chemicals. Short-term exposure (acute) to high levels of volatile organic compounds may cause eye, nose and throat irritation, headaches, nausea, vomiting, dizziness or worsening of asthma symptoms. Long-term exposure (chronic) to high levels of volatile organic compounds creates an increased risk of cancer, liver damage, kidney damage, and central nervous system damage. Thus, a need exists for removing volatile organic compounds from our air supplies.

### SUMMARY OF THE INVENTION

An air purification system formed from an adsorptive photocatalytic oxidation device and a method of regenerating the oxidation device is disclosed. The air purification system may be configured to be installed within an air duct of a central air handling system. The air purification system may also include a light source, such as an ultraviolet light emitted by an ultraviolet light source, to break down captured volatile organic compounds into elemental carbon dioxide and water vapor and to irradiate air moving past the ultraviolet light and surfaces to reduce contaminants. While the device 10 is described using ultraviolet light, other light sources such as LED lights or pulsed xenon flash lamps (i.e. pulsed light) can be used as well. The ultraviolet light source may be positioned to expose the adsorptive photocatalytic oxidation device to ultraviolet light emitted by the ultraviolet light source. The air purification system controls and reduces indoor related volatile organic compounds by first adsorbing the airborne contaminate into the adsorptive photocatalytic oxidation device, which may be an activated carbon honeycomb monolithic cell or other material that has gas phase adsorbing capabilities, and then breaking the volatile organic compound contaminate down via a photocatalytic oxidation process to free up the adsorbing media to further absorb additional airborne contaminates.

The air purification system may include a housing having one or more adsorptive photocatalytic oxidation devices. The housing may be formed from a generally rectangular box containing at least one adsorptive photocatalytic oxidation device, and the ultraviolet light source extends from the housing. A deflector may extend from the housing along the ultraviolet light source to deflect air through the adsorptive photocatalytic oxidation device and to deflect ultraviolet radiation emitted from the ultraviolet light source. The ultraviolet light source may be positioned to expose the adsorptive photocatalytic oxidation device to ultraviolet light emitted by the ultraviolet light source to break down captured volatile organic compounds into elemental carbon dioxide and water vapor and to irradiate air moving past the ultraviolet light and surrounding surfaces to reduce contaminants. The adsorptive photocatalytic oxidation device may be formed from an adsorption media. In one embodiment, the adsorption media may be an activated carbon monolithic material. The adsorptive photocatalytic oxidation device may be formed from a highly absorbent form of activated carbon configured in a low pressure drop honeycomb monolith. The media may be other highly adsorptive material as well. Also, the material may be constructed so as to be conductive. The air purification system may also include a coating of a regenerative photocatalyst blended within or coated onto the adsorptive photocatalytic oxidation device. The coating of a regenerative photocatalyst may be, but is not limited to being, an ultraviolet light reactive titanium dioxide based semiconductor photocatalyst. The photocatalyst can be doped or blended with materials to make it reactive with other light sources, such as visible light sources or sunlight or fluorescent lighting.

The air purification system may be installed in a central air handling system. In particular, the air purification system may be installed in an air duct extending therefrom; the housing and ultraviolet light source may be positioned in the air duct. The air purification system may also be installed within the air handling unit of the central air system as well. The adsorptive photocatalytic oxidation device is designed to capture volatile organic compounds, odors or other gas phase chemical or organic compounds in the air being passed through the air duct. The ultraviolet light may kill contaminants, including, but not limited to, algal, fungal, bacterial, and viral contamination. The ultraviolet light may also regenerate the adsorptive photocatalytic oxidation device.

An advantage of this invention is that the air purification system may remove volatile organic compounds from air being passed through the air purification system and may remove contaminants, such as, but not limited to, algal, fungal, bacterial, and viral contamination from the air and surfaces with the use of ultraviolet light.

Another advantage of this invention is that the ultraviolet light regenerates the adsorptive photocatalytic oxidation device through a photocatalytic oxidation process.

Yet another advantage of this invention is that the air purification system may be sold as a kit to retrofit currently existing central air handling systems.

These and other components are described in more detail below.

### BRIEF DESCRIPTION OF THE DRAWINGS

The accompanying drawings, which are incorporated in and form a part of the specification, illustrate embodiments of the presently disclosed invention and, together with the description, disclose the principles of the invention.

FIG. 1A is a front perspective view of an air purification system for removing volatile organic compounds and for removing contaminants from the air being moved by the air purification system.

FIG. 1B is a back perspective view of the air purification system for removing volatile organic compounds and for removing contaminants from the air being moved by the air purification system.

FIG. 2A is a side perspective view with partial cutaway sections of the air purification system installed in a duct of a central air handling system immediately downstream of an air handler.

FIG. 2B is a schematic of the air purification system with a central air handling system having additional features.

FIG. 3A is a detailed perspective view of an adsorptive photocatalytic matrix of the air purification system as shown in FIG. 1B.

FIG. 3B illustrates a single cell of the adsorptive photocatalytic matrix.

FIG. 4 is a diagram of the adsorption process of the air purification system.

FIG. 5 is a diagram of the regeneration process of the air purification system.

FIG. 6 is a graph of an air purification system test on the removal of toluene from air.

FIG. 7 is a graph of an air purification system test on the removal of odors from air.

FIG. 8 is a graph of an air purification system test showing the amount of volatile organic compounds removed from air.

FIG. 9 is a schematic representation of an illustrative embodiment of an ionizer/ion generator positioned within the inside of the housing unit.

FIG. 10A is a schematic representation illustrating a mechanism of action of the enhanced regenerative photo-catalyst composition.

FIG. 10B is an additional representation illustrating a mechanism of action of the enhanced regenerative photo-catalyst composition.

### DETAILED DESCRIPTION OF THE INVENTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

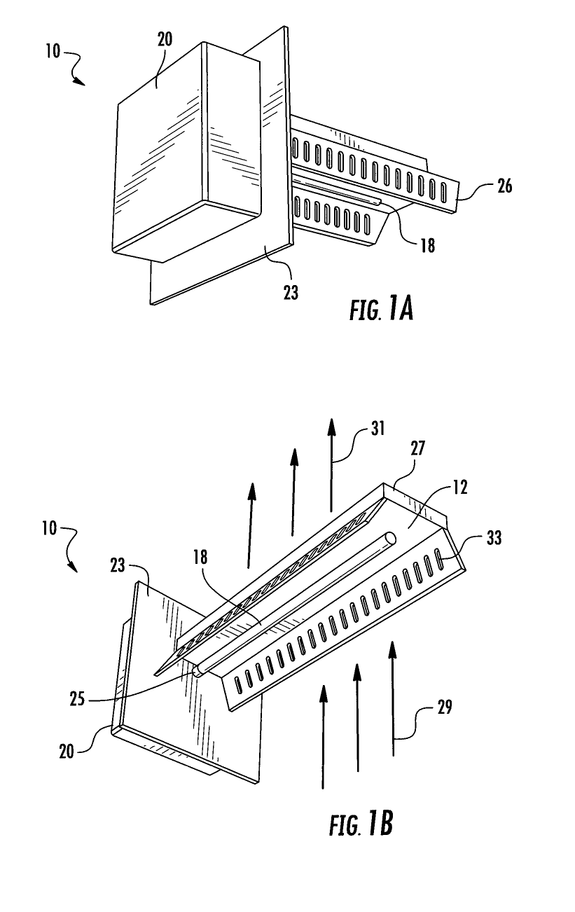

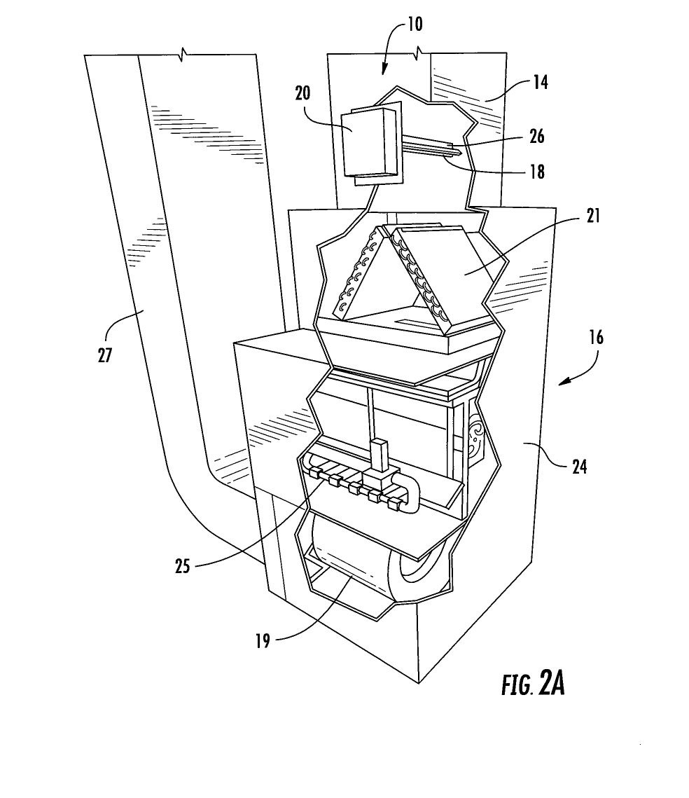

As shown in FIGS. 1A, 1B, and 2A, an air purification system, referred to as air purification system 10, having an adsorptive photocatalytic oxidation member 12 is disclosed. The air purification system 10 may be configured to be installed within an air duct 14 of a central air handling system 16. The air purification system 10 includes an electromagnetic radiation source, preferably an ultraviolet light emitted by an ultraviolet light source 18 to break down captured volatile organic compounds into elemental carbon dioxide and water vapor and to irradiate air moving past the ultraviolet light and local surfaces to reduce contaminants. The ultraviolet light source 18 is preferably positioned to irradiate microorganisms in the air stream and to expose the adsorptive photocatalytic oxidation member 12 to ultraviolet light emitted by the ultraviolet light source 18. The UV light, therefore, irradiates the moving air stream, helping to reduce airborne bacteria, viruses and allergens, and organic odors. Use of the UV light may further eliminate any build up of mold from within air ducts 14 or other parts of the air conditioning unit, such as the air handlers 24.

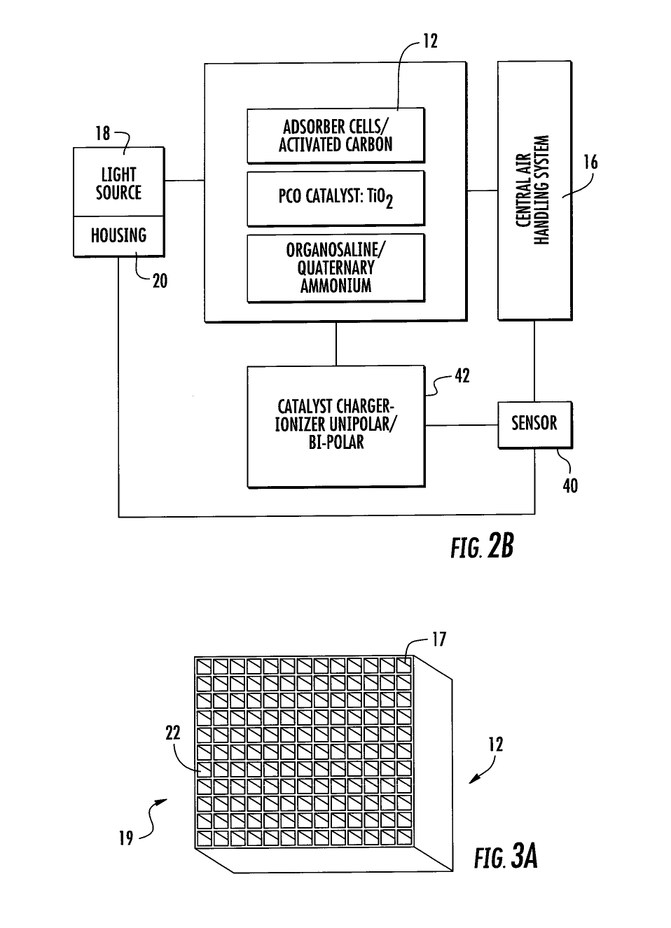

The air purification system 10 controls and reduces indoor related volatile organic compounds by first adsorbing the airborne contaminate into the adsorptive photocatalytic oxidation member 12 and then breaking the volatile organic compound contaminate down via a photocatalytic oxidation process. The adsorptive photocatalytic oxidation member 12 may be an activated carbon honeycomb monolithic cell 19, see FIGS. 3A and 3B. The honeycomb monolithic cell may be made of other absorbing media types such as alumina ceramic (aluminum oxide). As illustrated in FIG. 3A, the activated carbon honeycomb monolithic cell 19 may be composed of an absorption media matrix having a plurality of individual units 17. This media can also be charged to increase its affinity to uptake airborne contaminates.

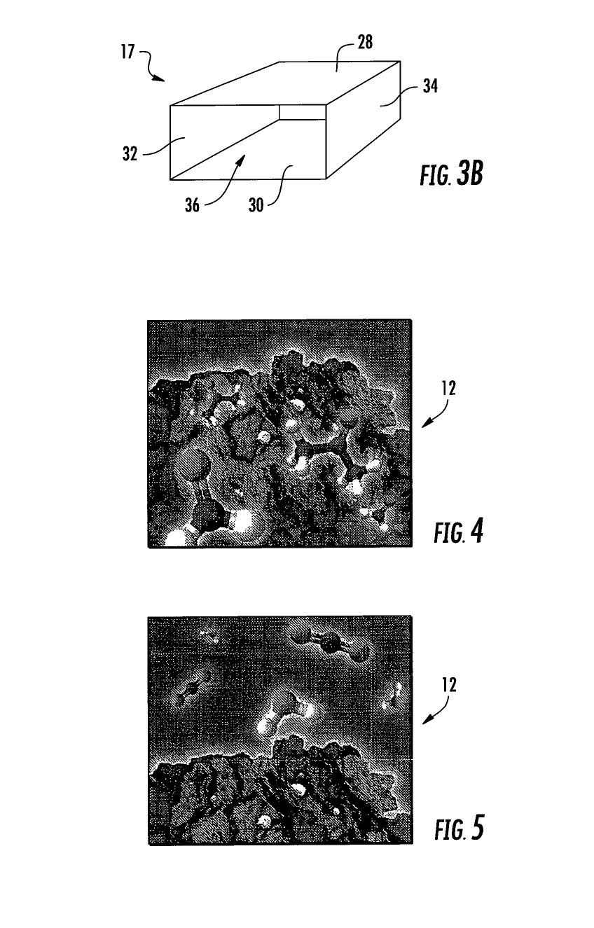

The air purification system 10 is designed to help sterilize the air and reduce indoor odors, microorganisms and volatile organic compound contamination from indoor air. By using an adsorption media, the air purification system 10 captures volatile organic compounds, as shown in FIG. 4, and then reduces the captured constituents through an innovative photocatalytic oxidation process that breaks down the captured volatile organic compounds into elemental carbon dioxide and water vapor. The individual units 17, may be, but are not limited to being an adsorptive activated carbon which can be regenerated. In use, the media first adsorbs and holds the VOC chemicals into “sites” or holes in the carbon. The light then catalytically reacts via the TiO₂ catalyst on the surface and the UV light. The catalytic process then breaks down the held or captured chemical to an elemental form, thus freeing up the carbon site to adsorb once again. This process is a room temperature process. The individual units 17, therefore, may be continuously regenerated for on-going air treatment, as shown in FIG. 5. Also, the material can be constructed of other highly adsorptive materials or can be additionally ionically charged to increase its ability to absorb more VOC's/microorganisms than when not charged.

Housing 20 may be sized and shaped to contain one or more adsorptive photocatalytic oxidation members 12. The housing may be formed from resilient materials such as, but not limited to, metals and plastics. The housing 20 may contain a generally rectangular box 21 secured to support structure 23. The generally rectangular box 21 contains the internal functional components (not shown) of the light electromagnetic radiation source, such as UV ballast and a power source. Accordingly, when a user secures a UV light bulb 18 into the electrical contact 25, UV light is provided. Extending from the support structure 23 is an elongated member 27 sized and shaped to extend over and cover UV light source 18. As shown in FIG. 1B, the adsorptive photocatalytic oxidation member 12 is housed and secured under the elongated member 27. This places the adsorptive photocatalytic oxidation member 12 downstream of the UV light source, see arrows 29 and 31 indicating the direction of air flow through the air purification system 10. In one embodiment, at least one light source is positioned perpendicular to air flow through said system. This allows light to enter into each individual unit 17. A deflector 26, which contains a plurality of slotted openings 33, may extend from the elongated member 27 along the ultraviolet light source to deflect air through the adsorptive photocatalytic oxidation device 12 and to deflect ultraviolet radiation emitted from the ultraviolet light source 18. The deflector 26 may have any appropriate configuration. In at least one embodiment, the deflector 26 may be a three-sided device generally forming a U-shaped device. The deflector 26 may be formed from resilient materials such as, but not limited to, metals and plastics.

The adsorptive photocatalytic oxidation member 12 may be formed from an adsorption media, which may be a highly adsorptive activated carbon honeycomb monolithic media. The adsorptive photocatalytic oxidation member 12 preferably includes regenerative photocatalyst. As an illustrative example (FIG. 3B), each individual cell unit 17 includes a coating 22 of the regenerative photocatalyst on the adsorptive photocatalytic oxidation member 12. Referring to FIG. 3B, the individual unit 17 is shown. The individual unit 17 is formed of two opposing walls 28 and 30, forming the top and bottom, and two opposing side walls 32 and 34. Each of the walls 28, 30, 32, and 34 may be coated on the interior surfaces, the exterior surfaces, or both the interior and exterior surfaces with the coating 22. Individual cell 17 is preferably open at both ends, exposing the interior 36 to light. Individual cell 17 however, may be designed to have at least one closed end. The regenerative photocatalyst coating 22 may be an ultraviolet reactive titanium dioxide based semi-conductor photocatalyst or other form of precious metal semiconductor photocatalyst material. While the adsorptive photocatalytic oxidation member 12 is illustrated with rows of 6 cells, it may be constructed of multiple rows of individual units 17 for a higher uptake rate.

Preferably, the regenerative photocatalyst coating may be a novel two component composition which forms a new chemical molecule, referred to generally as an enhanced regenerative photocatalyst, with both photocatalytic action and surface binding and antimicrobial properties. The enhanced regenerative photocatalyst composition comprises 1) an organosilane, preferably an organosilane quaternary ammonium, and 2) a photocatalyst, such as titanium dioxide TiO₂. Other photocatalysts may include Zinc Oxide (ZnO), tungsten trioxide (WO₃), Zirconium dioxide (ZiO₂), or cadmium sulfide (CdS). The composition is believed to be effective by utilizing one or more characteristics. The organosilane imparts positive charge on the composition. The positive charge attracts the negatively charged microbe or contaminate VOCs. The organosilane component is further believed to puncture and chemically kill the microbe and breakdown the contaminate VOCs. Finally, the titanium dioxide (TiO2) is believed to reduce pathogens or contaminate VOCs through the reactive oxidative stress (ROS) process.

In general, organosilane chemistry involves monomeric silicon chemicals known as silanes. A silane that contains at least one carbon-silicon bond (Si—C) structure is known as an organosilane. The organosilane molecule (Formula 1) has three key elements:

X—R—Si(OR′)3 (Formula 1)

Wherein: X is a non-hydrolyzable organic moiety. This moiety can be reactive toward another chemical (e.g., amino, epoxy, vinyl, methacrylate, sulfur) or nonreactive (e.g., alkyl; wherein OR′ is a hydrolyzable group, like an alkoxy group (e.g., methoxy, ethoxy isopropoxy) or an acetoxy group that can react with various forms of hydroxyl groups present in mineral fillers or polymers and liberates alcohols (methanol, ethanol, propanol) or acid (acetic acid). These groups can provide the linkage with inorganic or organic substrate; and wherein R is a spacer, which can be either an aryl or alkyl chain, typically propyl-. [R′=Methyl, Ethyl, Isopropy, R=Aryl or Alkyl (CH2)ₙ with n=0, 1 or 3].

Typical organosilane quaternary compounds in accordance with the present invention include, but are not limited to: 3-(trimethoxysilyl)propyldimethyloctadecyl ammonium chloride; 3-(trimethoxysilyl)propyldidecylmethyl ammonium chloride; 3-(trimethoxysilyl)propyltetradecyidimethyl ammonium chloride; 3-(trimethoxysilyl)propyldimethylsoya ammonium chloride; 3-(trimethoxysilyl)propyldimethyloleyl ammonium chloride; 3-(trimethoxysilyl)propyloctadecyl ammonium chloride; 3-(trimethoxysilyl)propyloleyl ammonium chloride; 3-(trihydroxysilyl)propyldimethyloctadecyl ammonium chloride; and 3-(trimethoxysilyl)propyldocosane ammonium chloride; 3-(trimethoxysilyl)propylmethyldi(decyl) ammonium chloride; 3-chhlorpropyltrimethoxysilane; octadecyltrimethoxysaline; per fluorooctyltriethoxysaline; benzalkonium chloride; glycine betaine; or siltrane compounds (alkanoalmine in combination with organosilicon quaternary ammonium) as described in U.S. Pat. No. 5,064,613.

Preferably, the enhanced regenerative photocatalyst composition is formed with titanium dioxide (TiO₂) in the nano particle form. Accordingly, reference to TiO₂ includes titanium dioxide nanoparticles, including TiO₂, anatase grade. TiO₂ can be doped, or incorporated with other elements, or dopants, to make it more responsive to a wider range of light including, but not limited to zinc oxide, zirconium dioxide, nitrogen, silicone, silver (Ag), carbon, iron, or copper.

As such, the enhanced regenerative photocatalyst composition is both an organosilane surface binding molecule and a photocatalytic molecule. The composition forms a multi-functional, anti-microbial biocide/contaminate VOCs degrader having several of the following characteristics: 1) a silane base which serves to combine the molecules together and to other surfaces, such as to the surface of the Activated Carbon monolith or cells; 2) the molecule contains a positively charged component for attracting microbes or contaminate VOCs towards the molecule; 3) a long chain for mechanically and chemically puncturing, as well as chemically neutralizing microbes and degrades/breaks down contaminate VOCs; and 4) a photocatalytically activating molecule, creating a reactive oxygen and hydroxyl radical environment which oxidizes microbes and degrades contaminate VOCs and catalyzes chemical compounds via the light activated catalytic process.

TABLE 1
Example 1. Composition of the enhanced regenerative photocatalyst
applied to the adsorptive photocatalytic oxidation device.

| Component | Concentration |
|---|---|
| Organosilane | The concentrated composition is |
| | composed of 1 part Organosilane to |
| | 2 parts light activated photocatalyst |
| Photocatalyst | |
| Water | QS w/concentrated composition to |
| | desired effective concentration |

TABLE 2
Example 2. Composition of the enhanced regenerative photocatalyst
applied to the adsorptive photocatalytic oxidation device.

| Component | Concentration |
|---|---|
| Quaternary ammonium | The concentrated composition is |
| | composed of 1 part quaternary |
| | compound to 2 parts light activated |
| | photocatalytic agent |
| Light activated | |
| photocatalytic agent | |
| Water | QS w/concentrated |
| | composition to desired |
| | effective concentration |

TABLE 3
Example 3. Composition of the enhanced regenerative photocatalyst
applied to the adsorptive photocatalytic oxidation device.

| Component | Concentration |
|---|---|
| Organosilane | Concentrated composition is |
| quaternary ammonium | composed of 1 part organosilane |
| | quaternary ammonium to 2 parts TiO2 |
| Titanium dioxide | |
| Water | QS w/concentrated composition |
| | to desired effective |
| | concentration |

TABLE 4
Example 4. Composition of the enhanced regenerative photocatalyst
applied to the adsorptive photocatalytic oxidation device.

| Component | Concentration |
|---|---|
| 3-(Trihydroxysilyl)pro- | Concentrated composition is |
| pyldimethyloctadecyl | composed of 1 part |
| ammonium chloride | 3-(Trihydroxysilyl) |
| | propyldimethyloctadecyl |
| | ammonium chloride to 2 |
| | parts Sol Gel Titanium |
| | Dioxide |
| Sol Gel Antase form | |
| of Titanium Dioxide | |
| Water | QS w/concentrated |
| | composition to |
| | desired effective |
| | concentration |

Preferably, the enhanced regenerative photocatalyst composition is composed of 2 parts TiO₂ to 1 part organosilane quaternary compound form a concentrated compound. The concentrated compound is diluted approximately 20:1 for an applied concentration dosage of approximately 1000-1250 ppm.

As shown in FIG. 1, the ultraviolet light source 18 may extend from the housing 20. The ultraviolet light source 18 may be positioned to expose the adsorptive photocatalytic oxidation device 12 to ultraviolet light emitted by the ultraviolet light source 18 to break down captured volatile organic compounds into elemental carbon dioxide and water vapor and to irradiate air moving past the ultraviolet light 18 to reduce contaminants.

As shown in FIG. 2A, the air purification system 10 may be used to clean air passing through a HVAC (heating, ventilation, and air condition system) 16. The air handler 24 may contain, for example, an A/C coil 21, a blower 19 and a furnace element 25 connected to a return air duct 27. The housing 20 and ultraviolet light source 18 may be positioned in the supply air duct (plenum) 14. While the air purification system 10 is described as using a UV light source, other light sources may be used, including but not limited to, a mercury vapor style of light source, light emitting diodes (LED), or xenon bulbs. The ultraviolet light source 18 may produce light in the UV-C germicidal spectrums, such as 254 nm. This spectrum is effective in sterilizing microbial contaminates. When placed in the air duct 14, the ultraviolet light source 18 may be positioned to provide the interior space of the central air handling system 16 with a way of controlling surface microbial contamination within the interior components of the unit. The ultraviolet light source 18 may produce light in the UV-C spectrum for the purpose of sterilization of microbial contamination.

FIG. 2B illustrates the air purification system 10 designed to include electromagnetic irradiation over a spectrum. For example, air purification system 10 may include a light source 18 designed to provide a UV spectrum (100 nm-700 nm) using various, different light sources, such as UV-C lamps, UV Spectrum or discrete spectrum LED or pulsed UV lamps. The light source may be configured to be dynamic, i.e. a dimmable UV lamp or LED may be used as an energy saving means. A light source producing light waves in the range of above 360 nm to 480 nm may be used. In such case, the titanium dioxide may be doped with various other elements to make it more responsive to the wider range of light; in particular by doping the photocatalyst, the light spectrums in the visible ranges above 400 nanometers can become effective, whereas undoped photocatalysts are only affective up to 365 nm. The elements include, but are not limited to zinc oxide, zirconium dioxide, nitrogen, silicone, silver (Ag), carbon, iron, or copper. As an illustrative example, nitrogen doped titanium dioxide/quaternary ammonium composition allows the catalyst functionality to work in spectrums above 365 nm, such as at 405 nm. By working in spectrums above 400 nm, this allows the photocatalyst to work with visible light sources, such as fluorescent lights, sunlight or low cost LEDS. Use of LEDS in the UV spectrum are more expensive than visible range LEDS. Systems that can function in different light ranges are more cost effective and are more functional over a wider range of light sources beyond the UV based ones.

FIG. 2B further illustrates additional features that may be included in air purification system 10. These features may be used individually or in any combination with the other features of the air purification system 10 as previously described. The air purification system 10 may include a sensor, such as a VOC sensor or other indoor odor or airborne chemical gas type sensor(s) 40 to control the intensity of the unit as a function of contaminate load. The VOC sensor can be utilized to detect various levels of odors and chemical gases in the indoor living space in which the air purification is be utilized. If the sensor senses a predetermined level of such contaminates, the air purification system 10 can be charged by an enhanced ionically charged catalyst 42. The sensor may also be functionally connected to other parts of the device/system or a main control unit so as to, for example, turn on/off the light source or HVAC blower. The enhanced, ionically charged catalyst mechanism is designed to direct positively or negatively charged ions in close proximity towards the individual activated carbon cells. As the airborne molecules come into close proximity to the ion charge point(s), odor molecules or chemical gas molecules are charged and become attracted towards the grounded, negatively or neutrally charged activated carbon cell mechanism. This allows for increases in uptake rates or absorbance capabilities of the cells, and also increases in the affinity to further absorb VOCs, or microorganisms, above that of uncharged carbon cells. The activated carbon cells can be constructed out of materials that enhance the electrical potential of the structure, such as graphite, aluminum oxide or other conductive metal based ceramic material. By making the cells conductive, this further enhances the ionic charging capability of the cells to further enhance the uptake of charged materials.

Preferably, the charge catalyst 42 is an ionizer/ion generator 43 positioned, for example, inside the housing 20. FIG. 9 illustrates a schematic representation of an illustrative embodiment of the placement of the ionizer/ion generator 43 inside of housing unit 20. In addition to the ionizer/ion generator 43, the housing 20 may contain a light power source, illustrated herein as the UV power supply (ballast) 45. The UV power supply powering the UV light lamp 18. The ionizer/ion generator 43 may be designed to provide an ion distribution bar or ion points extending away from the housing 20 to allow for charged ions 49 to be distributed/directed to predetermined areas, such as the areas at, near or below the UV lamp 18. A control unit 51 may be configured to actuate the ionizer 43. The control unit 51 may be configured in such a manner to switch the ionizer 43 on or off to produce a predetermined amount of ions, and/or may be connected to a sensor to produce ions when the levels of containments in an area reach a certain set point. Preferably, the ions are distributed with the direction of air flow through the system. While the charged ions 49 are illustrated as positive ions, negative ions can be generated as well. Although the ionizer 43 is preferably placed within the housing 20, the ionizer 43 may be positioned in other areas associated with Applicant's system. The intention of the ionizer 43 is to impart an ionic charge (anion charge or cation charge) to the moving airstream in order to charge gas particles or microbials so they will be attracted to the activated carbon matrix to be photocatalytically destructed. An illustrative example of an ionizer 43 may include a power supply that creates the high voltage potential necessary to create the ions, an ion distribution header for creating a single point or multiple points to distribute the ions into the air, and an ionization point which can be comprised of a needle point or a carbon fiber brush that creates the point in which the high voltage potential is converted to electron potential for creating the ions in the air. The ions can be positive charged (cation) or negative charged (anion) or a bias of both (i.e. 40% positive, 60% negative) of either potential. Applicant's device or system is configured to create and direct the ions toward the gas particles with a bias or opposite potential to attract them towards the absorber cells, i.e. the carbon matrix. Traditional ionizers create hydrogen or oxygen ions, superoxide ions, or peroxides to go out into the living environment and oxidize and coagulate particles and gases to either cause them to settle out in the environment or to be filtered out (such as the case with portable air purifiers). In this approach, airborne contaminates adsorb into the carbon cells to mitigate them in-situ or on the surface of the cells instead of in the air or the living space. Even though we use hydroxyl radicals as well, this hydroxyl radical process is a surface mediated process and is never airborne.

In use, the air purification system 10 may be installed in the air duct 14 of one or more central air handling systems 16. As odors and chemical contaminates, such as volatile organic compounds (VOCs) including ethanol, acetone, acetaldehyde, and formaldehyde, circulate through the air handling system 16, the air purification system 10 may utilize a highly adsorptive activated carbon monolithic media 12 that captures these contaminates, removing them from the air stream, much like a sponge absorbs water.

Activated carbon adsorption is an effective method for removing gaseous contaminates. Although carbon is an extremely effective way of adsorbing airborne contaminates, it has a finite capacity to adsorb these contaminates. To overcome this limitation, the activated carbon monolithic media 12 of the air purification system 10 has been coated with the regenerative photocatalyst. This UV reactive titanium dioxide (TiO₂) based semi-conductor photocatalyst, when exposed to ultraviolet light, becomes highly reactive and attacks the chemical bonds of adsorbed volatile organic compounds and bio-aerosol pollutants or microorganisms, thereby reducing these adsorbed gaseous chemicals and biological contaminants to carbon dioxide (CO2), and water vapor (H2O). Other forms of precious metal semiconductor photocatalyst material may be used as a catalyst. This process is referred to as photocatalytic oxidation and is highly effective at breaking down complex volatile organic compounds and microorganisms. The air purification system 10 uses the absorption capabilities of carbon to adsorb airborne volatile organic compounds and the catalytic oxidation ability of UV photocatalytic oxidation technology to regenerate the carbon. As the airborne molecules come into close proximity to the ion charge point, odor molecules or chemical gas molecules are charged and become attracted towards the grounded, negatively or neutrally charged activated carbon cell mechanism. This allows for increases in uptake rates or adsorbance capabilities of the cells, and also increases in the affinity to absorb VOCs above that of uncharged carbon cells.

During the off cycles of the central air handling system 16, the self regenerating photocatalytic process of the air purification system 10 breaks down the captured contaminates and frees up the activated carbon honeycomb monolithic cell to be able to capture additional airborne volatile organic compounds and odors. In addition to the ability of the air purification system 10 to adsorb airborne volatile organic compounds, the ultraviolet light source 18 plays an important role in disinfecting and deodorizing the indoor air of any bacteria, viruses, molds and allergens, reducing indoor air related allergies and illness. In addition, the ultraviolet light source 18 helps to maintain the cleanliness of the air handling system by shining direct onto the ductwork, cooling coils, heat strips and blowers that are prone to have mold growth. During use, the ultraviolet light source 18 may irradiate ultraviolet light continuously or at intervals. The ultraviolet light may prevent growth and kill existing microbial contamination.

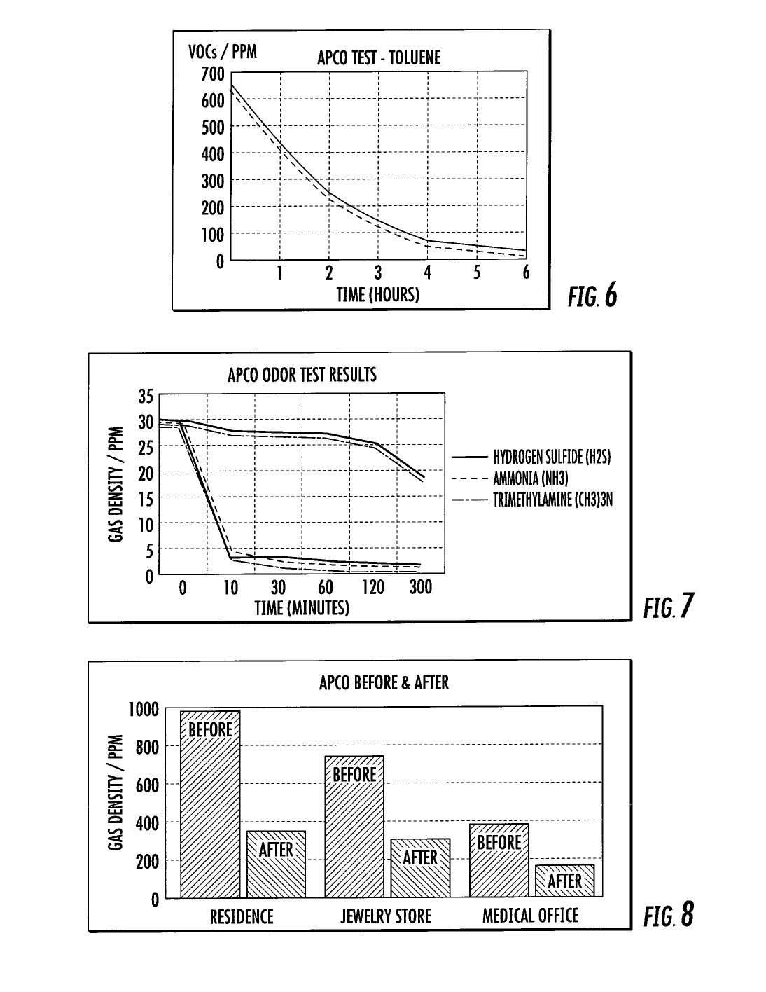

FIGS. 6-8 illustrate the effectiveness of the air purification system 10 which does not use enhanced regenerative photocatalyst composition. As shown in FIG. 6, the air purification system 10 may remove volatile organic compounds from air. In particular, air containing volatile organic compounds in amounts approaching 650 parts per million (ppm) may be reduced in about four hours to about 75 ppm. Further, the air purification system 10 may remove volatile organic compounds from amounts approaching 650 parts per million (ppm) in about six hours to about 35 ppm. The UV light source in the test was a 254 nm germicidal UV-C spectrum quartz hot filament. The photocatalytic oxidation device was a monolithic adsorptive cell with adsorption media and TiO2 photo-catalyst. There were 16 cells per inch, 250 square meters/gm, a bulk density of 1.44 gm/cm2, a pressure drop of less than 0.005 in water column (w.c.) at 400 fpm, volatile organic compound activity of between 40% and 60% adsorption per pass, and a maximum operating temperature of 400 degrees Fahrenheit.

FIG. 7 is a graph of the results of removing odor from air with the air purification system 10. Within about 10 minutes of passing air through the air purification system 10, the concentrations of ammonia and trimethylamine were reduced from about 30 ppm to about 3 ppm and about 4.5 ppm, respectively. Hydrogen Sulfide was removed from air from a starting concentration of about 30 ppm to about 18 ppm over about 300 minutes.

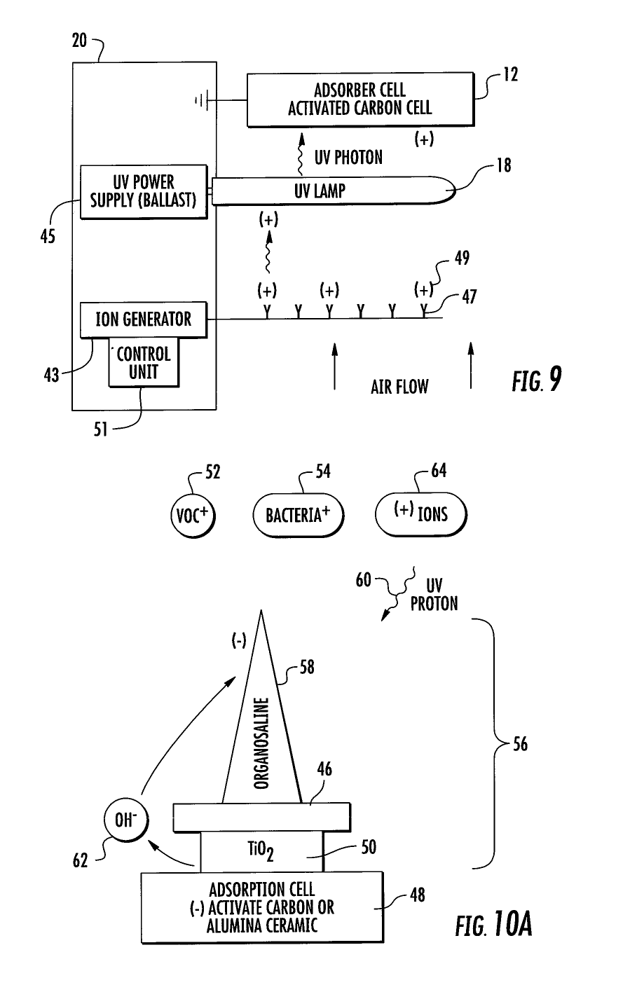

FIG. 8 shows a graph of the results of the air purification system 10 of removing volatile organic compounds from air. In particular, the air purification system 10 may reduce volatile organic compounds in residences, jewelry stores, and medical offices from between about 57 percent and about 62 percent.

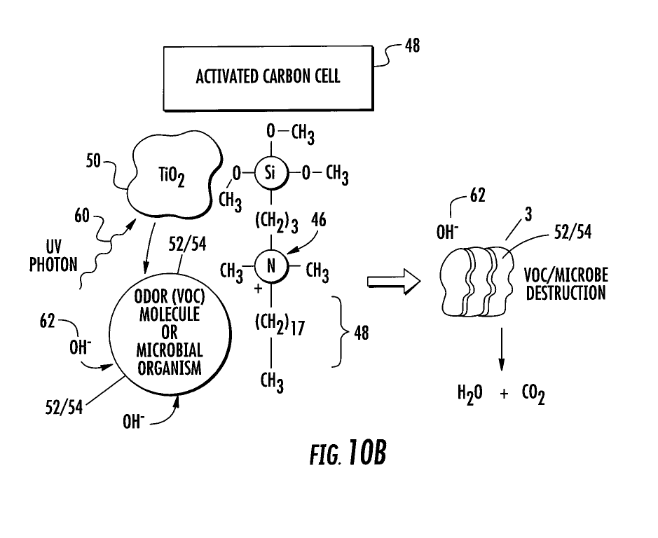

It is anticipated that use of the enhanced regenerative photocatalyst composition with regards to various VOCs or living contaminants with or without the ionizer will provide enhanced benefits when compared to a system that uses only activated carbon and titanium dioxide. FIG. 10A and FIG. 10B are schematic representations illustrating a mechanism of action and possible basis for anticipated benefits associated with the use of enhanced regenerative photocatalyst composition. The combination of the organosilane/quaternary ammonium chemistry with a photocatalytic material is believed to enhance reactivity with airborne VOC's and microorganisms over that of the catalyst or activated carbon alone. Activated carbon has an inherent affinity to attract and adsorb contaminates via Van der Waals affects. TiO2 has been found to have certain adsorptive properties due to the PCO materials having some porosity. However, the addition of the organosilane/quaternary ammonium will provide additional attractive forces to the activated Carbon/TiO2 matrixes to increase adsorption. As illustrated in FIGS. 10A and 10B, the organosilane component 46 is bound to the surface of the activated carbon 48 and/or the TiO2 50 during the manufacturing process via the silane base of the organosilane. The positive charge of the nitrogen molecule of the quaternary ammonium portion of the organosilane can attract VOC's 52 or microorganisms, such as bacteria 54 to the new chemical organosilane/titanium dioxide/carbon structure 56. In turn, the long chain carbon molecules (represented by number 58 FIG. 10A) of the chemical structure acts to spear microbiologicals (bacteria, etc), killing them through puncturing or a cellular lysis process. It is speculated that these long chain carbon molecules may also exhibit an additional ability to “hold” chemicals to their surfaces in order for the photocatalytic process to break them down. Such photocatalytic process results from titanium dioxide being exposed to UV light 60, which in turn produce free radicals (hydroxyl radicals), illustrated herein as HO⁻ molecules, see 62. Additionally, it is believed that the enhanced regenerative photocatalyst composition may further provide an “electrocution” affect on micro-organisms. This effect is based on the difference in the positive charge of the nitrogen molecule and the negative charge of the microorganism. The positive charge affect of the nitrogen may attract chemical compounds that otherwise would not normally be attracted to activated carbon, thus attracting and holding them to the structure for the photocatalytic process to break them down. Accordingly, the charged nitrogen and carbon molecule holding process is speculated to attract and hold other chemical compounds over that of activated carbon alone. Additionally, the system may utilize the charged ions 64 to enhance the effect. In this system, absorption of the airborne contaminates into the carbon cells mitigate them in-situ or on the surface of the cells, providing a surface mediated process.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. An air purification system, comprising:
at least one adsorptive photocatalytic oxidation housing storing an adsorptive photocatalytic oxidation member comprising an enhanced regenerative photocatalyst composition, said enhanced regenerative photocatalyst composition comprising at least one organosilane and at least one photocatalyst; and
at least one light source positioned to expose said adsorptive photocatalytic oxidation member to light emitted by said light source, whereby said light exposure converts volatile organic compounds into elemental carbon dioxide and water vapor and irradiates air moving past said light source and local surfaces to reduce contaminants.

2. The air purification system according to claim 1, wherein said adsorptive photocatalytic oxidation member is an activated carbon honeycomb monolithic material.

3. The air purification system according to claim 2, wherein said activated carbon honeycomb monolithic is ionically charged.

4. The air purification system according to claim 3, wherein said enhanced regenerative photocatalyst composition is coated onto said an activated carbon honeycomb monolithic material.

5. The air purification system according to claim 1 wherein said at least one organosilane is a quaternary ammonium.

6. The air purification system according to claim 5 wherein said at least one a photocatalyst is titanium dioxide.

7. The air purification system according to claim 1 wherein said light source is an ultraviolet light source.

8. The air purification system according to claim 1 wherein said at least one photocatalyst is doped with a dopant.

9. The air purification system according to claim 8 wherein said dopant is zinc oxide, zirconium dioxide, nitrogen, silicone, silver, carbon, iron, or copper.

10. The air purification system of claim 9 wherein said dopant is nitrogen.

11. The air purification system according to claim 1 wherein said system includes a light source configured to produce light in the range of between 110 nm to 700 nm.

12. The air purification system according to claim 1 wherein said at least one light source is positioned perpendicular to air flow through said system.

13. The air purification system according to claim 1 wherein said enhanced regenerative photocatalyst composition comprises 2 parts of titanium dioxide to 1 part organosilane quaternary compound.

14. The air purification system according to claim 1 further including a deflector.

15. The air purification system according to claim 1 further including a sensor.

16. The air purification system according to claim 1 further including an ionizer.

17. An air purification system, comprising:
a housing having at least one adsorptive photocatalytic oxidation member with at least one outer surface exposed;
an ultraviolet light source positioned to expose said adsorptive photocatalytic oxidation device to ultraviolet light emitted by the ultraviolet light source to break down captured volatile organic compounds into elemental carbon dioxide and water vapor and to irradiate air moving past said ultraviolet light and surfaces to reduce contaminants;
a coating of a regenerative photocatalyst on the at least one adsorptive photocatalytic oxidation device;
wherein the at least one adsorptive photocatalytic oxidation device is formed from an adsorption media; and
wherein the coating of a regenerative photocatalyst is an ultraviolet reactive titanium dioxide based semi-conductor photocatalyst.

18. The air purification system of claim 16, wherein said regenerative photocatalyst includes an organolsilane.
